(19) United States
(12) Patent Application Publication
(10) Pub. No.: US 2008/0058285 A1
(43) Pub. Date: Mar. 6, 2008

(54) Colored perfumed compositions comprising at least one alkyl-beta,beta'-diphenylacrylate or alkyl alpha-cyano-beta,beta'-diphenylacrylate compound and at least one soluble organic UV-A filter

(75) Inventors: Elisabeth Picard-Lesboueyries, Velizy (FR); Liliane Lecomte Desgranges, Thiais (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS, FRANCE.

(21) Appl. No.: 11/892,942

(22) Filed: Aug. 28, 2007

Related U.S. Application Data
(60) Provisional application No. 60/899,628, filed on Feb. 6, 2007.

(30) Foreign Application Priority Data
Aug. 30, 2006 (FR) 06/07619

(51) Int. Cl. A61K 8/33; A61K 31/695; A61K 8/49; A61K 8/58; A61Q 13/00

(52) U.S. Cl. 514/63; 512/6

(57) ABSTRACT

Topically applicable colored and perfumed compositions, well suited for perfuming such human keratinous substrates as the skin, lips and/or integuments, contain, formulated into a cosmetically acceptable medium: a) at least 1% by weight of a perfuming substance; b) at least one volatile alcohol and/or volatile silicone oil; c) at least one coloring agent which is soluble in said medium; d) at least 0.2% by weight of at least one alkyl β,β′-diphenylacrylate or alkyl α-cyano-β,β′-diphenylacrylate compound; and a) at least one organic UV-A filter which is soluble in said medium; b) and, optionally, water.

### CROSS-REFERENCE TO PRIORITY/PROVISIONAL APPLICATIONS

This application claims priority under 35 U.S.C. § 119 of FR 06/07619, filed Aug. 30, 2006, and of U.S. Provisional Application No. 60/899,628, Feb. 6, 2007, each hereby expressly incorporated by reference and each assigned to the assignee hereof.

### BACKGROUND OF THE INVENTION

1. Technical Field of the Invention

The present invention relates to colored perfumed compositions comprising, formulated into a cosmetically acceptable medium:

a) at least 1% by weight with respect to the total composition weight of at least one perfuming substance;

b) at least one volatile alcohol and/or volatile silicone oil;

c) at least one coloring agent which is soluble in said medium;

d) at least 0.2% by weight with respect to the total composition weight of at least one alkyl β,β′-diphenylacrylate or alkyl α-cyano-β,β′-diphenylacrylate compound with respect to the total composition weight; and

e) at least one organic UV-A filter which is soluble in said medium;

f) and optionally water.

2. Description of Background and/or Related and/or Prior Art:

A perfume is an association of various odoriferous substances which evaporate at different times. Each perfume exhibits what is termed a “top note” which is the odor which diffuses first when the perfume is applied or on opening the receptacle containing it, a “heart or middle note” which corresponds to the complete perfume (emitted for several hours after the “top note”), and a “bottom note” which is the most persistent odor (emitted for several hours after the “heart note”). The tenacity of the bottom note corresponds to how long a perfume lingers.

Human beings have always sought to perfume themselves and their surroundings, both to mask strong and/or disagreeable odors and to provide a pleasant odor.

Perfume is routinely incorporated into many products or compositions, in particular cosmetic and dermatological compositions such as splashes, toilet water, eau de parfum, aftershave lotions and skin care water. For reasons regarding aesthetics and manufacturing costs, the perfume concentrate is colored by adding an effective quantity of soluble coloring agent to the support for the formulation (generally alcoholic or hydroalcoholic) rather than tinting or lacquering the bottle which constitutes a more expensive industrial operation. The color developed in those perfumed formulations must remain stable both over time and when exposed to light. In general, a screening system and/or an antioxidant system is added.

As one example, WO 2005/042828 describes colored perfuming compositions comprising, as a stabilizing system, a piperidinol derivative (i.e., Tris(tetramethylhydroxypiperidinol) citrate—Tinoguard Q) associated with an organic UV filter selected from among dibenzoylmethane derivatives, cinnamates, camphor derivatives and s-triazines. In particular, Example 6 describes toilet water comprising a perfume, a coloring agent, 0.1% of a mixture of Butylmethoxydibenzoylmethane and octocrylene, a piperidinol type stabilizer.

In addition, WO 00/25370 describes colored toilet waters stabilized by a particular benzotriazole compound and/or a particular triazine, for example Sodium Benzotriazolyl Butylphenol Sulfonate such as the product marketed under the trademark “TINOGUARD HS” by CIBA-GEIGY, Benzotriazolyl dodecyl p-Cresol such as the product marketed under the trademark “TINOGUARD TL” by CIBA-GEIGY, the product marketed under the trademark “CIBAFAST H LIQUID” by CIBA-GEIGY, or Bumetrizole such as the product marketed under the trademark “TINOGUARD AS” by CIBA-GEIGY.

However, certain of those screening and/or antioxidant systems are ineffective and/or tend to denature the desired color.

There exists a need for colored perfuming products which do not exhibit the disadvantages of the prior art products, and in particular a need for colored perfuming products the color of which remains stable over time and under the influence of light.

### SUMMARY OF THE INVENTION

It has now surprisingly been determined that the above objectives can be achieved by means of an association of at least one alkyl β,β′-diphenylacrylate or alkyl α-cyano-β,β′-diphenylacrylate compound and at least one organic UV-A filter which is soluble in the perfumed medium.

This discovery constitutes the basis of the invention.

The present invention features colored perfumed compositions comprising, formulated into a cosmetically acceptable medium:

a) at least 1% by weight with respect to the total composition weight of at least one perfuming substance;

b) at least one volatile alcohol and/or volatile silicone oil;

c) at least one coloring agent which is soluble in said medium;

d) at least 0.2% by weight with respect to the total composition weight of at least one alkyl β,β′-diphenylacrylate or alkyl α-cyano-β,β′-diphenylacrylate compound with respect to the total composition weight; and

e) at least one organic UV-A filter which is soluble in said medium;

f) and, optionally water.

This invention also features incorporating at least 0.2% by weight of at least one alkyl β,β′-diphenylacrylate or alkyl α-cyano-β,β′-diphenylacrylate compound and at least one soluble UV-A organic filter as defined above in a colored perfumed composition comprising, formulated into a cosmetically acceptable medium:

a) at least 1% by weight with respect to the total composition weight of at least one perfuming substance;

b) at least one volatile alcohol and/or volatile silicone oil;

c) at least one coloring agent which is soluble in said medium;

d) and optionally water;

as a color stabilizing agent for said composition.

The present invention also features a cosmetic regime or regimen for perfuming human keratinous substances, in particular the skin, lips, and integuments, comprising the topical application of a composition as defined above to the keratinous substances.

### DETAILED DESCRIPTION OF BEST MODE AND SPECIFIC/PREFERRED EMBODIMENTS OF THE INVENTION

The term “perfuming substance” or “perfume” means any perfume or aroma which is capable of releasing an agreeable odor.

Preferably, the quantity of perfuming substance(s) is from 1% to 30% by weight, more preferably 2% to 25% by weight with respect to the total composition weight.

The term “cosmetically acceptable medium” in the compositions of the invention means a non-toxic medium which is capable of being applied to the skin (including the inside of the eyelids), the lips, the nails or the hair of human beings.

The term “organic UV-A filter” means any compound comprising at least one chromophoric group which is capable of filtering UV radiation in the UV-A radiation range, namely with a wavelength in the range 320 nm to 400 nm.

The term “soluble in the medium” means any compound which is capable of being completely dissolved in a phase comprising at least one volatile alcohol and/or at least one volatile silicone oil and optionally water in addition.

The term “volatile alcohol” means any compound comprising at least one alcohol function having a vapor pressure at 20° C. of more than 17.5 mm of mercury.

The term “oil” according to the present invention means a fat which is insoluble in water and which is liquid at ambient temperature and atmospheric pressure.

The term “volatile silicone oil” according to the present invention means any silicone compound which is capable of evaporating on contact with the skin or keratinous fiber in less than one hour, at ambient temperature and atmospheric pressure. The volatile compound is a volatile cosmetic compound which is liquid at ambient temperature, having a non-zero vapor pressure at ambient temperature and atmospheric pressure, especially having a vapor pressure of 0.13 Pa to 40,000 Pa (10⁻³ to 300 mm Hg), in particular 1.3 Pa to 13,000 Pa (0.01 to 100 mm Hg), and more particularly 1.3 Pa to 1300 Pa (0.01 to 10 mm Hg).

The perfuming substance contained in the compositions of the invention may derive from perfumes and aromas of natural or synthetic origin and mixtures thereof. Examples of perfumes and aromas of natural origin are flower extracts (lily, lavender, rose, jasmine, ylang-ylang), stems and leaves (patchouli, geranium, bitter leaf), fruits (coriander, anis, cumin, juniper), fruit skin (bergamot, lemon, orange), roots (angelica, celery, cardamom, iris, sweet flag), wood (pinewood, sandalwood, lignum vitae, pink cedar), herbs and graminaceae (tarragon, lemon grass, sage, thyme), needles and branches (spruce, fir, pine, dwarf pine), resins and balsams (galbanum, gum elemi, gum benzoin, myrrh, frankincense, opopanax).

Examples of perfuming substances of synthetic origin are compounds of the ester, ether, aldehyde, ketone, aromatic alcohol and hydrocarbon type.

Particular esters which are exemplary are benzyl acetate, benzyl benzoate, phenoxyethyl isobutyrate, p-tert-butylcyclohexyl acetate, citronellyl acetate, citronellyl formate, geranyl acetate, linalyl acetate, dimethylbenzylcarbinyl acetate, phenylethyl acetate, linalyl benzoate, benzyl formate, ethylmethylphenyl glycinate, alkylcyclohexyl propionate, styralyl propionate and benzyl salicylate.

An ether which is exemplary is benzylethylether.

Examples of aldehydes are linear alkanals containing 8 to 18 carbon atoms, citral, citronellal, citronellyloxyacetaldehyde, cyclamenaldehyde, hydroxycitronellal, lilial and bourgeonal.

Examples of ketones are ionones such as alpha-isomethylionone, and methylcedrylketone.

Aromatic alcohols and in particular terpenes which are exemplary are anethol, citronellol, eugenol, isoeugenol, geraniol, linalol, phenylethylalcohol and terpineol.

Particular hydrocarbons which are exemplary are terpenes. These compounds are usually in the form of a mixture of two or more of these odoriferous substances.

Furthermore, it is also possible to incorporate essential oils, components of aromas, such as sage, camomile, clove, melissa, mint, cinnamon leaf, lime blossom, juniper, vetiver, frankincense, galbanum, labolanum and lavender essences.

Preferably, the perfuming substance, used alone or as a mixture, is bergamot essence, dihydromyrcenol, lilial, lyral, citronellol, phenylethyl alcohol, alpha-hexylcinnamaldehyde, geraniol, benzylacetone, cyclamenaldehyde, linalol, ambroxane, indol, hedione, sandelice, lemon, mandarin and orange essences, allylamine glycolate, cyclovertal, lavender essence, sage essence, betadamascone, geranium essence, cyclohexyl salicylate, phenylacetic acid, geranyl acetate, benzyl acetate or rose oxide.

In a preferred embodiment of the invention, a mixture of various perfuming substances is used which in symphony creates a note which is pleasant to the consumer. Examples of known olfactory notes are hesperide perfumes, aromatics, floral perfumes, musks, fruity perfumes, spicy perfumes, oriental perfumes, oceanic perfumes, aquatic notes, chypre perfumes, woody perfumes, fougeres and mixtures thereof.

Alkyl β,β′-diphenylacrylates and alkyl α-cyano-β,β′-diphenylacrylates according to the present invention are selected from among those having the following formula (I): 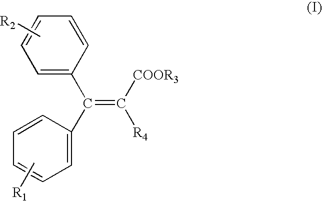
in which:

R₁ and R₂, which may be identical or different, are in the meta- or para-position and are selected from: hydrogen; a linear or branched C₁-C₈ alkoxy radical; and a linear or branched straight-chain C₁-C₄ alkyl radical;

R₃ is a linear or branched straight-chain C₁-C₁₂ alkyl radical;

R₄ is a hydrogen atom or a —CN radical.

More particularly preferred alkyl β,β′-diphenylacrylates and alkyl α-cyano-β,β′-diphenylacrylates in accordance with the present invention are 2-ethylhexyl α-cyano-β,β′-diphenylacrylate or ethyl α-cyano-β,β′-diphenylacrylate.

2-ethylhexyl α-cyano-β,β′-diphenylacrylate, also termed octocrylene, is known as a lipophilic filter which absorbs in the UV-B. It is commercially available and is marketed in particular under the trademark “UVINUL N 539” by BASF. It has the following formula: 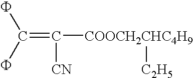
in which φ designates a phenyl radical.

Ethyl α-cyano-β,β′-diphenylacrylate, also known as etocrylene, is also a lipophilic filter which absorbs in the UV-B. It is commercially available and is marketed in particular under the trademark “UVINUL N 35” by BASF. It has the following formula: 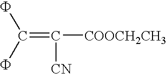
in which φ designates a phenyl radical.

2-ethylhexyl α-cyano-β,β′-diphenylacrylate is more particularly preferred.

Preferably, the alkyl β,β′-diphenylacrylate or alkyl α-cyano-β,β′-diphenylacrylate is present in the compositions of the invention in an amount of at least 0.2% to preferably 0.5% to 3% by weight, and more particularly 0.5% to 1.5% by weight with respect to the total composition weight.

Thus, when a sufficient quantity of the association of the invention is added to a colored perfumed composition, an increase in the stability of the color over time and under the effect of light is observed.

UV-A filters which are soluble in the composition medium may preferably be selected from among benzophenone derivatives, dibenzoylmethane derivatives, benzylidene camphor sulfonic derivatives such as those described in FR-2,528,420, and benzotriazole silicones such as those cited in EP-660,701.

Benzophenone derivatives which are soluble in the composition medium which are exemplary include:
Benzophenone-2 marketed under the trademark “UVINUL D50” by BASF,

Benzophenone-3 or Oxybenzone, marketed under the trademark “UVINUL M40” by BASF,

n-hexyl 2-(4-diethylamino-2-hydroxybenzoyl)benzoate, marketed under the trademark “UVINUL A+” by BASF or “UVINUL A+B” in the form of a mixture with octylmethoxycinnamate.

Benzylidene camphor sulfonic derivatives which are soluble in the composition medium which are exemplary include:

Terephthalylidene Dicamphor Sulfonic Acid marketed under the trademark “MEXORYL SX” by CHIMEX.

More particular benzotriazole silicones which are soluble in the composition medium which are exemplary include compounds having the following general formula (II): 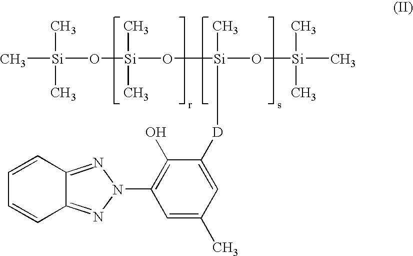
wherein: 0≦r≦15, preferably 0≦r≦10;
1≦s≦5, preferably 1≦s≦3;
   and in which D is the divalent radical: 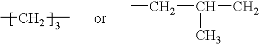
   and more particularly the compound Drometrizole Trisiloxane having formula (II) where r=0 and s=1 and 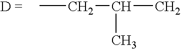
   marketed under the trademark “Silatrizole” by RHODIA CHIMIE.

A non-limiting list of dibenzoylmethane derivatives in accordance with the invention includes:
2-methyldibenzoylmethane;
4-methyldibenzoylmethane;
4-isopropyldibenzoylmethane;
4-tert-butyldibenzoylmethane;
2,4-dimethyldibenzoylmethane;
2,5-dimethyldibenzoylmethane;
4,4′-diisopropyldibenzoylmethane;
4,4′-dimethoxydibenzoylmethane;
4-tert-butyl-4′-methoxydibenzoylmethane;
2-methyl-5-isopropyl-4′-methoxydibenzoylmethane;
2-methyl-5-tert-butyl-4′-methoxydibenzoylmethane;
2,4-dimethyl-4′-methoxydibenzoylmethane;
2,6-dimethyl-4-tert-butyl-4′-methoxydibenzoylmethane.

Particular dibenzoylmethane derivatives of those mentioned above which are exemplary include 4-isopropyldibenzoylmethane, marketed under the trademark “EUSOLEX 8020” by MERCK, having the following formula:

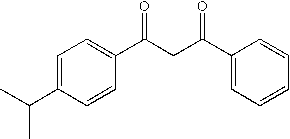

Particularly preferably, 4-(tert-butyl) 4′-methoxy dibenzoylmethane or Butyl Methoxy Dibenzoylmethane is used, marketed under the trademark “PARSOL 1789” by DSM; this filter has the following formula:

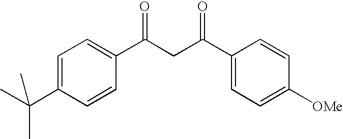

More particularly, the soluble UV-A filters in accordance with the invention are selected from the dibenzoylmethane type, more particularly 4-(tert-butyl) 4′-methoxy dibenzoylmethane or Butyl Methoxy Dibenzoylmethane.

The soluble UV-A filter or filters of the invention are preferably present in the compositions of the invention in an amount of 0.01% to 3% by weight, more particularly 0.5% to 1.5% by weight with respect to the total composition weight.

The cosmetically acceptable medium of the present invention contains at least one volatile alcohol and/or volatile silicone oil and optionally water. Preferably, the composition medium contains water in a quantity preferably of 0.01% to 50% to more preferably 0.5% to 25% by weight with respect to the total composition weight.

The volatile alcohols of the present invention are preferably selected from lower C₁-C₅ monoalcohols, and may be selected from among methanol, ethanol, propanol, isopropanol, n-butanol, isobutanol and t-butanol, more particularly ethanol.

The volatile alcohol or alcohols are preferably present in quantities of 40% to 80% to more preferably in quantities of 55% to 80% by weight with respect to the total composition weight.

Examples of volatile silicone oils are volatile linear or cyclic silicone oils, especially those with a viscosity of ≦6 centistokes (6.10⁻⁶ m²/s), and in particular containing 2 to 10 silicon atoms, said silicones optionally comprising alkyl or alkoxy groups containing 1 to 22 carbon atoms. Volatile silicone oils according to the invention which are particularly exemplary are octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

The volatile silicone oil or oils are preferably present in concentrations of 40% to 98.5%, preferably in concentrations of 10% to 80% by weight with respect to the total composition weight.

In accordance with one embodiment of the invention, one or more supplemental organic UV filters which are active in the UV-B region and which are soluble in the composition medium are also incorporated.

In particular, the organic UV filters are selected from among cinnamic derivatives, salicylic derivatives and cationic camphor alkylsulfate derivatives such as those described in FR 2 199 971, and mixtures thereof.

Examples of supplemental organic photoprotective agents are those designated below with their INCI names:

Cinnamic Derivatives:

Ethylhexyl Methoxycinnamate marketed in particular under the trademark PARSOL MCX by HOFFMANN LAROCHE,

Salicylic Derivatives:

Homosalate marketed under the trademark “Eusolex HMS” by Rona/EM Industries,

Ethylhexyl Salicylate marketed under the trademark “NEO HELIOPAN OS” by HAARMANN and REIMER,

Cationic Camphor Alkylsulfate Derivatives:

Camphor Benzalkonium Methosulfate, produced under the trademark “MEXORYL SO” by CHIMEX,

and mixtures thereof.

The supplemental photoprotective agents are preferably present in the compositions in quantities of 0.01% to 3% by weight and more preferably 0.5% to 1.5% by weight with respect to the total composition weight.

Preferred soluble coloring agents or colorants in accordance with the invention are hydrosoluble or hydrophilic coloring agents which include:

caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal red (CI 15850, CI 75470), Ext. Violet 2, Red 6-7, Ferric Ferrocyanide, Ultramarines, Acid Yellow 3/Yellow 10, Acid Blue 3, Yellow 10.

The soluble coloring agent or coloring agents of the invention are preferably present in quantities of 10⁻⁵% to 1% of the total composition weight, preferably 10⁻⁴% to 0.1% of the total composition weight.

Furthermore, the compositions of the invention may include any additive which is normally used in the perfume field, in particular selected from among antioxidants, cosmetic or dermatological active ingredients such as emollients or softeners such as sweet almond oil, apricot kernel oil, moisturizing agents such as glycerin, soothing agents such as α-bisabolol, allantoin, aloe Vera; vitamins, essential fatty acids, insect repellents, propellants, peptizing agents, fillers, nacres, glitter and mixtures thereof. When present in the compositions of the invention, these additives may be present in a quantity from 0.001% to 10%, preferably 0.01% to 5% by weight with respect to the total composition weight.

Examples of antioxidants are BHA (tert-butyl-4-hydroxyanisole), BHT (2,6-di-tert-butyl-p-cresol), and tocopherols such as vitamin E and its derivatives, such as tocopheryl acetate.

In another particular embodiment of the invention, the colored perfuming composition contains no color stabilizer other than the alkyl β,β′-diphenylacrylate or α-cyano-β,β′-diphenylacrylate compound/soluble UV-A filter association. Examples of perfume color stabilizers are Tris(tetramethylhydroxypiperidinol) citrate such as the product marketed under the trademark “TINOGUARD Q” by CIBA-GEIGY, Sodium Benzotriazolyl Butylphenol Sulfonate such as the product marketed under the trademark “TINOGUARD HS” by CIBA-GEIGY; Benzotriazolyl dodecyl p-Cresol such as the product marketed under the trademark “TINOGUARD TL” by CIBA-GEIGY, the product marketed under the trademark “CIBAFAST H LIQUID” by CIBA-GEIGY, Bumetrizole such as the product marketed under the trademark “TINOGUARD AS” by CIBA-GEIGY.

Clearly, one skilled in this art will be careful to select any complementary additives and/or their quantities so that the advantageous properties of the compositions of the invention are not or are not substantially altered by the envisaged addition.

The present invention is not only applicable to colored perfuming products but is also applicable to skin care products, skin treatment products, including those for treatment of the scalp and the lips, containing an odoriferous substance. The compositions of the invention may thus constitute a composition for perfuming, for skin care, for treating keratinous substances and in particular may be in the form of a splash, toilet water, eau de parfum, aftershave lotion, skin care water or silicone or hydrosilicone skin care oil. Same may also be in the form of a two-phase perfumed lotion (toilet water phase/hydrocarbon oil and/or silicone oil phase).

This invention also features a cosmetic regime or regimen for perfuming keratinous substances in human beings and in particular the skin, lips and integuments, comprising the topical application of the composition as defined above to the keratinous substances.

The compositions of the invention may be formulated via known methods which are generally employed in the field of perfumed and colored lotions.

The compositions of the invention in the form of lotions may be packaged into bottles. They may also be applied in the form of fine particles using pressurization devices. The devices of the invention are well known to one skilled in the art and include non-aerosol pumps or atomizers, aerosol receptacles including a propellant and aerosol pumps using compressed air as the propellant. The latter are described in U.S. Pat. Nos. 4,077,441 and 4,850,517.

The aerosol compositions of the invention generally contain conventional propellants such as the hydrofluorinated compounds dichlorodifluoromethane or difluoroethane, dimethylether, isobutane, n-butane, propane or trichlorofluoromethane.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

The following compositions were prepared:

| | Ex. 1 | | | | |
|---|---|---|---|---|---|
| Ingredients | (invention) | Ex. 2 | Ex. 3 | Ex. 4 | Ex. 5 |
| Oriental type perfume (*) | 6.99 | 6.99 | 6.99 | 6.99 | 6.99 |
| BHT | 0.025 | 0.025 | 0.025 | 0.025 | 0.025 |
| Yellow 5 | 0.00094 | 0.00094 | 0.00094 | 0.00094 | 0.00094 |
| Green 5 | 0.00016 | 0.00016 | 0.00016 | 0.00016 | 0.00016 |
| Red 4 | 0.00041 | 0.00041 | 0.00041 | 0.00041 | 0.00041 |
| Octocrylene | 0.5 | — | — | — | 0.5 |
| Butyl Methoxy | 0.5 | 0.16 | — | — | — |
| Dibenzoylmethane | | | | | |
| Ethylhexyl salicylate | — | 0.15 | — | — | — |
| Octylmethoxycinnamate | — | 0.69 | — | 0.20 | — |
| n-Hexyl 2-(4-diethylamino-2- | — | — | — | 0.10 | — |
| hydroxybenzoyl) benzoate | | | | | |
| Tris(tetramethylhydroxypiper- | — | — | 0.03 | — | — |
| idinol) citrate (Tinoguard Q) | | | | | |
| Benzophenone-3 | | 0.2 | 0.2 | — | |
| Ethanol | 71.25 | 71.25 | 71.25 | 71.25 | 71.25 |
| Water | qsp 100 | qsp 100 | qsp 100 | qsp 100 | qsp 100 |

(*) perfume from the Orientals family, characterized by: its top notes of the pomegranate, carambola, amaretto type; its heart notes of the jasmine, peony, red hibiscus type; its bottom notes of the tonka bean, myrrh, Perou oil type.

| | Ex. 6 | | |
|---|---|---|---|
| Ingredients | (invention) | Ex. 7 | Ex. 8 |
| Fruity floral type perfume (**) | 15.00 | 15.00 | 15.00 |
| BHT | 0.0059 | 0.0059 | 0.0059 |
| Red 4 | 0.000091 | 0.000091 | 0.000091 |
| Octocrylene | 0.58 | — | 0.58 |
| Butyl Methoxy | 0.58 | 0.58 | — |
| Dibenzoylmethane | | | |
| Ethanol | 71.36 | 71.36 | 71.36 |
| Water | qsp 100 | qsp 100 | qsp 100 |

(**) Perfume from the fruity-floral family characterized by: its top notes of the pear and rose pepper type; its heart notes of the rose otto, violet type; its bottom notes of the heliotrope and sandalwood type.

Each formula was packaged into a standard 50 ml glass bottle.

Each bottle was placed in a non-refrigerated solar simulator for a period of 16 hours using the following protocol:

Principle:

The principle of these tests consisted of exposing the test products to a source of radiant light with a well-defined spectral distribution and a well-quantified energy output.

Xenon lamps are light sources in routine use, emitting through:

a platinum-coated quartz filter which deflects infrared and eliminates it from the top of the apparatus;

and a glass filter, which by absorbing short-wavelength UV radiation can simulate the radiation received behind a display window.

Equipment:

A CPS Sun-test apparatus was used:

the illumination, provided by a xenon lamp from 300 nm and 800 nm, was fixed at 765 W/m² (value set by the manufacturer);

optical filtration was provided by a quartz filter with an IR coating and a special display window glass.

The color of each bottle before and after exposure to light was then observed with the naked eye and using a MINOLTA CM3600-d spectrophotometer. The color obtained was quantified. The results, given as means, were expressed in the (L*, a*, b*) system in which L* is brightness, a* is the red-green axis (−a*=green, +a*=red) and b* is the yellow-blue axis (−b*=blue, +b*=yellow). Thus, a* and b* express the color of the composition.

Results:

| | | | a * | | | | | |
|---|---|---|---|---|---|---|---|---|
| | L * | | before & | | b * | | Color observed with | |
| | before & after | | after | | before & after | | the naked eye, before | |
| Compositions | exposure | | exposure | | exposure | | & after exposure | |
| Ex 1(inv) | 91.96 | 94.15 | −0.02 | −3.20 | 37.41 | 33.10 | Orangey | Orangey |
| Ex 2 | 89.95 | 93.95 | −0.57 | −7.45 | 39.95 | 31.86 | Orangey | Discolored |
| | | | | | | | | yellow |
| Ex 3 | 92.29 | 96.65 | −0.6 | −1.6 | 37.96 | 7.13 | Orangey | Discolored |
| Ex 4 | 92.11 | 91.58 | −1.46 | −8.76 | 40.12 | 27.12 | Orange | Yellow |
| Ex 5 | | | 0.45 | −4.43 | 36.95 | 9.37 | Orange | Yellow |
| Ex 6 (inv) | 92.27 | 97.14 | 1.14 | 1.07 | 4.11 | 4.12 | Pale pink | Pale pink |
| Ex 7 | | | 1.41 | −1.34 | 4.02 | 3.01 | Pale pink | Discolored |
| Ex 8 | 98.97 | 100.2 | 1.95 | −0.61 | 2.61 | 1.54 | Pale pink | Discolored |

Examples 1 and 6 of the colored perfumes of the invention comprising the octocrylene/butylmethoxydibenzoylmethane association exhibited a stable color after 16 hours of exposure in the solar simulator, in contrast to Examples 2 to 5 and 7-8.

### Example 9

### Hydrosilicone Perfume

| Ingredients | Quantities |
|---|---|
| Fruity floral type perfume in | 15.00 |
| accordance with Examples 6 to 8 | |
| BHT | 0.0058 |
| Red 4 | 0.000091 |
| Dimethicone (DOW CORNING | 7.44 |
| FLUID 200 1.5 cst) | |
| Octocrylene | 0.58 |
| Butyl Methoxy | 0.58 |
| Dibenzoylmethane | |
| Ethanol | 71.36 |
| Water | qsp 100 |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, colored and perfumed composition comprising, formulated into a cosmetically acceptable medium therefor:
a) at least 1% by weight with respect to the total weight of the composition of at least one perfuming substance;
b) at least one volatile alcohol and/or volatile silicone oil;
c) at least one coloring agent which is soluble in said medium;
d) at least one alkyl β,β′-diphenylacrylate or alkyl α-cyano-β,β′-diphenylacrylate compound in a quantity of at least 0.2% by weight with respect to the total weight of the composition; and
e) at least one UV-A filter which is soluble in said medium;
f and, optionally, water.

2. The colored and perfumed composition as defined by claim 1, in which the quantity of perfuming substance ranges from 1% to 30% by weight with respect to the total weight of the composition.

3. The colored and perfumed composition as defined by claim 1, in which the alkyl β,β′-diphenylacrylate or the alkyl α-cyano-β,β′-diphenylacrylate is selected from among those having the following formula (I): 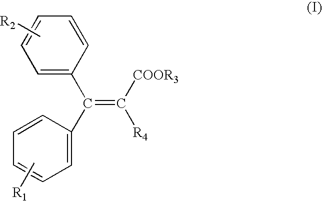
in which:
R₁ and R₂, which may be identical or different, are in the meta- or para-position and are selected from: hydrogen; a linear or branched C₁-C₈ alkoxy radical and a linear or branched straight-chain C₁-C₄ alkyl radical;
R₃ is a linear or branched straight-chain C₁-C₁₂ alkyl radical;
R₄ is a hydrogen atom or a —CN radical.

4. The colored and perfumed composition as defined by claim 3, in which the alkyl β,β′-diphenylacrylate or alkyl α-cyano-β,β′-diphenylacrylate is selected from 2-ethylhexyl α-cyano-β,β′-diphenylacrylate and ethyl α-cyano-β,β′-diphenylacrylate.

5. The colored and perfumed composition as defined by claim 4, in which the alkyl β,β′-diphenylacrylate or alkyl α-cyano-β,β′-diphenylacrylate is 2-ethylhexyl α-cyano-β,β′-diphenylacrylate.

6. The colored and perfumed composition as defined by claim 1, in which the UV-A filter or filters which are soluble in the composition medium are selected from the group consisting of benzophenone derivatives, dibenzoylmethane derivatives, benzylidene camphor sulfonic derivatives and benzotriazole silicones.

7. The colored and perfumed composition as defined by claim 6, comprising at least one benzophenone derivative selected from among:
Benzophenone-2;
Benzophenone-3;
n-hexyl 2-(4-diethylamino-2-hydroxybenzoyl)benzoate.

8. The colored and perfumed composition as defined by claim 6, comprising Terephthalylidene Dicamphor Sulfonic Acid.

9. The colored and perfumed composition as defined by claim 6, comprising at least one benzotriazole silicone selected from among compounds having the following general formula (II): 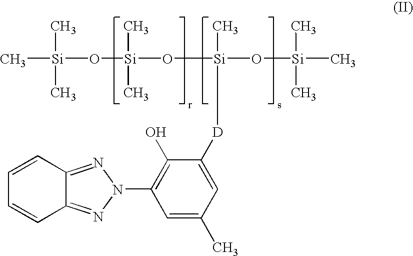
wherein: 0≦r≦15,
1≦s≦5,
and in which D is the divalent radical:

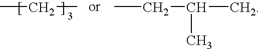

10. The colored and perfumed composition as defined by claim 9, comprising Drometrizole Trisiloxane having formula (II) where r=0 and s=1 and

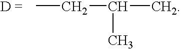

11. The colored and perfumed composition as defined by claim 6, comprising at least one dibenzoylmethane derivative selected from 4-isopropyldibenzoylmethane and 4-(tert-butyl) 4′-methoxy dibenzoylmethane.

12. The colored and perfumed composition as defined by claim 11, comprising 4-(tert-butyl) 4′-methoxy dibenzoylmethane.

13. The colored and perfumed composition as defined by claim 1, in which the soluble UV-A filter or filters are present in an amount of 0.01% to 3% by weight with respect to the total weight of the composition.

14. The colored and perfumed composition as defined by claim 1, in which the alkyl β,β′-diphenylacrylate or alkyl α-cyano-β,β′-diphenylacrylate is present in an amount of 0.5% to 3% by weight with respect to the total weight of the composition.

15. The colored and perfumed composition as defined by claim 1, in which the cosmetically acceptable medium contains water in a quantity of 0.01% to 50% by weight with respect to the total weight of the composition.

16. The colored and perfumed composition as defined by claim 1, comprising at least one volatile alcohol selected from among the lower C₁-C₅ monoalcohols.

17. The colored and perfumed composition as defined by claim 16, in which the at least one volatile alcohol comprises ethanol.

18. The colored and perfumed composition as defined by claim 1, comprising at least one volatile alcohol present in a quantity of 40% to 80% by weight with respect to the total weight of the composition.

19. The colored and perfumed composition as defined by claim 1, comprising at least one volatile linear or cyclic silicone oil having a viscosity of ≦6 centistokes (6×10⁻⁶ m²/s), and containing 2 to 10 silicon atoms, said silicones optionally comprising alkyl or alkoxy radicals having 1 to 22 carbon atoms.

20. The colored and perfumed composition as defined by claim 19, in which the volatile silicone oils are selected from the group consisting of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

21. The colored and perfumed composition as defined by claim 19, in which the volatile silicone oils are present in quantities of 40% to 98.5% by weight with respect to the total weight of the composition.

22. The colored and perfumed composition as defined by claim 1, further comprising one or more supplemental organic UV filters which are active in the UV-B region and which are soluble in the composition medium.

23. The colored and perfumed composition as defined by claim 22, in which the complementary organic UV-B filters are selected from the group consisting of cinnamic derivatives, salicylic derivatives, cationic benzylidene camphor alkylsulfate derivatives and mixtures thereof.

24. The colored and perfumed composition as defined by claim 23, in which the complementary organic UV filters are selected from among:
Ethylhexyl Methoxycinnamate;
Homosalate;
Ethylhexyl Salicylate;
Camphor Benzalkonium Methosulfate;
and mixtures thereof.

25. The colored and perfumed composition as defined by claim 22, in which the supplemental UV-B photoprotective agent or agents are present in quantities of 0.01% to 3% by weight with respect to the total weight of the composition.

26. The colored and perfumed composition as defined by claim 1, in which the coloring agent or coloring agents are present in quantities of 10⁻⁵% to 1% by weight with respect to the total weight of the composition weight.

27. The colored and perfumed composition as defined by claim 1, further comprising at least one additive selected from the group consisting of antioxidants, cosmetic or dermatological active ingredients, emollients or softeners, moisturizing agents, glycerin, soothing agents, vitamins, essential fatty acids, insect repellents, propellants, peptizing agents, fillers, nacres, glitter and mixtures thereof.

28. The colored and perfumed composition as defined by claim 1, formulated as a splash, toilet water, eau de parfum, aftershave lotion, skin care water, silicone or hydrosilicone skin care oil or two-phase lotion.

29. The colored and perfumed composition as defined by claim 1, comprising a lotion packaged in a bottle, a non-aerosol pump or an aerosol receptacle.

30. A cosmetic regime or regimen for perfuming human keratinous substrates, comprising topically applying thereon a thus effective amount of the colored and perfumed composition as defined by claim 1.

31. The cosmetic regime or regimen as defined by claim 30, said human keratinous substrates comprising the skin, lips and/or integuments.
